SEQUENCE LISTING (1 sequence)

SEQ ID NO: 1 - DNA, 70 bp, artificial sequence
  Synthetic sequence comprising a Vitellogenin- gene Estrogen Response Element
  misc_feature (1)..(5): 5/6 bases from 5′ end of cleaved Spe I site
  misc_feature (6)..(11): Xho I site
  misc_feature (27)..(32): Spacer Bgl II site
  misc_feature (70)..(70): 3′ overhang C from Afl II cleavage
ctagtctcgagaggtcactgtgacctagatctaggtcactgtgacctagatctaggtcactgtgacctac

(19) United States
(12) Patent Application Publication
(10) Pub. No.: US 2007/0004713 A1
(43) Pub. Date: Jan. 4, 2007

(54) Therapeutic benimidazole compounds

(76) Inventors: Bernard Barlaam, Reims (FR); Steven Dock, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/433,746

(22) PCT Filed: Dec. 7, 2001
(86) PCT No.: PCT/SE01/02725

§ 371 Date: Nov. 6, 2003

Related U.S. Application Data
(60) Provisional application No. 60/251,773, filed on Dec. 7, 2000.
(60) Provisional application No. 60/251,776, filed on Dec. 7, 2000.

(30) Foreign Application Priority Data
Jan. 2, 2001 (SE) 0100008-2
Jan. 2, 2001 (SE) 0100009-0

(51) Int. Cl. A61K 31/541; A61K 31/5377; A61K 31/496; A61K 31/454; A61K 31/427; A61K 31/422; A61K 31/4184; C07D 417/02; C07D 413/02; C07D 403/02

(52) U.S. Cl. 514/227.8; 514/234.2; 514/254.06; 514/320; 514/394; 548/307.4; 544/60; 544/139; 544/370; 546/199; 548/181; 548/304.7; 514/365; 514/374

(57) ABSTRACT

The invention relates to novel compounds having general formula (I) are useful as selective ER-β ligands in the treatment or prophylaxis of Alzheimer's disease, anxiety disorders, depressive disorders, osteoporosis, cardiovascular disease, rheumatoid arthritis or prostate cancer.

### TECHNICAL FIELD

The present invention is directed to a series of ligands, and more particularly to estrogen receptor-β ligands which have better selectivity than estrogen for the estrogen receptor-β over the estrogen receptor-α, as well as to methods for their production and use in the treatment of diseases related to the estrogen receptor-β, specifically, Alzheimer's disease, anxiety disorders, depressive disorders, osteoporosis, cardiovascular disease, rheumatoid arthritis, or prostate cancer.

### BACKGROUND

Estrogen-replacement therapy (“ERT”) reduces the incidence of Alzheimer's disease and improves cognitive function in Alzheimer's disease patients (Nikolov et al. Drugs of Today, 34(11), 927-933 (1998)). ERT also exhibits beneficial effects in osteoporosis and cardiovascular disease, and may have anxiolytic and anti-depressant therapeutic properties. However, ERT shows detrimental uterine and breast side effects that limit its use.

The beneficial effects of ERT in post-menopausal human women is echoed by beneficial effects of estrogen in models relevant to cognitive function, anxiety, depression, bone loss, and cardiovascular damage in ovariectomized rats. Estrogen also produces uterine and breast hypertrophy in animal models reminiscent of its mitogenic effects on these tissues in humans.

The beneficial effects of ERT in post-menopausal human women is echoed by beneficial effects of estrogen in models relevant to cognitive function, anxiety, depression, bone loss, and cardiovascular damage in ovariectomized rats. Specifically, experimental studies have demonstrated that estrogen effects the central nervous system (“CNS”) by increasing cholinergic function, increasing neurotrophin/neurotrophin receptor expression, altering amyloid precursor protein processing, providing neuroprotection against a variety of insults, and increasing glutamatergic synaptic transmission, among other effects. The overall CNS profile of estrogen effects in pre-clinical studies is consistent with its clinical utility in improving cognitive function and delaying Alzheimer's disease progression. Estrogen also produces mitogenic effects in uterine and breast tissue indicative of its detrimental side effects on these tissues in humans.

The estrogen receptor (“ER”) in humans, rats, and mice exists as two subtypes, ER-α and ER-β, which share about a 50% identity in the ligand-binding domain (Kuiper et al. Endocrinology 139(10) 4252-4263 (1998)). The difference in the identity of the subtypes accounts for the fact that some small compounds have been shown to bind preferentially to one subtype over the other (Kuiper et al.).

In rats, ER-β is strongly expressed in brain, bone and vascular epithelium, but weakly expressed in uterus and breast, relative to ER-α. Furthermore, ER-α knockout (ERKO-α) mice are sterile and exhibit little or no evidence of hormone responsiveness of reproductive tissues. In contrast, ER-β knockout (ERKO-β) mice are fertile, and exhibit normal development and function of breast and uterine tissue. These observations suggest that selectively targeting ER-β over ER-α could confer beneficial effects in several important human diseases, such as Alzheimer's disease, anxiety disorders, depressive disorders, osteoporosis, and cardiovascular disease without the liability of reproductive system side effects. Selective effects on ER-β-expressing tissues (CNS, bone, etc.) over uterus and breast could be achieved by agents that selectively interact with ER-β over ER-α.

It is a purpose of this invention to identify ER-β-selective ligands that are useful in treating diseases in which ERT has therapeutic benefits.

It is another purpose of this invention to identify ER-β-selective ligands that mimic the beneficial effects of ERT on brain, bone and cardiovascular function.

It is another purpose of this invention to identify ER-β-selective ligands that increase cognitive function and delay Alzheimer's disease progression.

### SUMMARY OF THE INVENTION

This present invention is directed to compounds having the generic structure: 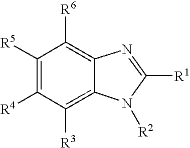
These compounds are ER-β-selective ligands, which mimic ERT, but lack undesirable side effects of ERT and are useful in the treatment or prophylaxis of Alzheimer's disease, anxiety disorders, depressive disorders, osteoporosis, cardiovascular disease, rheumatoid arthritis or prostate cancer.

These compounds particularly satisfy the formula:
(K_{iαA}/K_{iβA})/(K_{iαE}/K_{iβE})>1,
preferably:
(K_{iαA}/K_{iβA})/(K_{iαE}/K_{iβE})>30,
more preferably:
(K_{iαA}/K_{iβA})/K_{iαE}/K_{iβE})>100,
wherein K_{iαA} is the Kᵢ value for the ligand in ER-α; K_{iβA} is the Ki value for the ligand in ER-β; K_{iαE} is the Kᵢ value for estrogen in ER-α; and K_{iβE} is the Kᵢ value for estrogen in ER-β.

### DETAILED DESCRIPTION OF THE INVENTION

The compounds of the instant invention are ER-β-selective ligands of the structure: 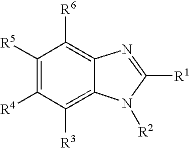
wherein:

R¹ is C₁₋₈alkyl, phenyl, benzyl or a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈alkyl, phenyl, benzyl or heterocycle is substituted by 1, 2 or 3 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; and wherein the phenyl, benzyl or heterocycle is additionally substituted by 0, 1 or 2 substituents selected from C₁₋₆alkyl, phenyl or benzyl;

R² is H, C₁₋₆alkyl, —(CH₂)ₘphenyl, —(CH₂)ₘnaphthyl or —(CH₂)ₘheterocycle, wherein the heterocycle is a 5- or 6-membered ring heterocycle containing 1, 2 or 3 heteroatoms each independently selected from O, N and S and additionally having 0 or 1 oxo groups and 0 or 1 fused benzo rings, wherein the C₁₋₆alkyl, —(CH₂)ₘphenyl, —(CH₂)ₘnaphthyl or —(CH₂)ₘheterocycle are substituted with 0, 1 or 2 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl;

R³ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R³ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;

R⁴ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl;

R⁵ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl;

R⁶ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R⁶ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;

R^{a} is H, C₁₋₆alkyl, C₁₋₃haloalkyl, phenyl or benzyl;

m is 0, 1, 2 or 3; and

In another embodiment, in addition to the above limitations, R¹ is C₁₋₈alkyl or a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈alkyl or heterocycle is substituted by 1, 2 or 3 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; and wherein the heterocycle is additionally substituted by 0, 1 or 2 substituents selected from C₁₋₆alkyl, phenyl or benzyl.

In another embodiment, in addition to the above limitations, R² is C₁₋₆alkyl, —(CH₂)ₘphenyl, —(CH₂)ₘnaphthyl or —(CH₂)ₘheterocycle, wherein the heterocycle is a 5- or 6-membered ring heterocycle containing 1, 2 or 3 heteroatoms each independently selected from O, N and S and additionally having 0 or 1 oxo groups and 0 or 1 fused benzo rings, wherein the —(CH₂)ₘphenyl, —(CH₂)ₘnaphthyl or —(CH₂)ₘheterocycle are substituted with 0, 1 or 2 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; and the C₁₋₆alkyl is substituted with 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

In another embodiment, in addition to the above limitations, R³ is C₁₋₆alkyl, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R³ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

In another embodiment, in addition to the above limitations, R⁴ is —R^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl.

In another embodiment, in addition to the above limitations, R⁵ is —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl.

In another embodiment, in addition to the above limitations, R⁶ is C₁₋₆alkyl, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R⁶ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

In another embodiment, in addition to the above limitations, R¹ is phenyl or benzyl, wherein the phenyl or benzyl is substituted by 1, 2 or 3 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl. In a more specific embodiment, R¹ is 4-hydroxyphenyl substituted by 0, 1 or 2 substituents selected from —OR^{a}, —SR^{a}, —NR^{a}R^{a}, —CO₂R^{a}, —OC(═O)R^{a}, —(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; and wherein the phenyl or benzyl is additionally substituted by 0, 1 or 2 substituents selected from C₁₋₆alkyl, phenyl or benzyl.

In another embodiment, in addition to the above limitations, R⁴ is OH.

In another embodiment, in addition to the above limitations; R⁵ is OH.

Particularly useful compounds have any of the above embodiments and also satisfy the equation:
(K_{iαA}/Ki_{βA})/(K_{iαE}/K_{iβE})>100, wherein

K_{iαA} is the Kᵢ value for the agonist in ER-α;

K_{iβA} is the Kᵢ value for the agonist in ER-β;

K_{iαE} is the Kᵢ value for estrogen in ER-α; and

K_{iβE} is the Kᵢ value for estrogen in ER-β.

Another aspect of the invention is the use of any of the above compound embodiments for the manufacture of a medicament for the treatment or prophylaxis of Alzheimer's disease, anxiety disorders, depressive disorders, osteoporosis, cardiovascular disease, rheumatoid arthritis or prostate cancer.

Another aspect of the invention is a method of using any of the above compound embodiments in the treatment or prophylaxis of Alzheimer's disease, anxiety disorders, depressive disorders (including postpartum and post-menopausal depression), osteoporosis, cardiovascular disease, rheumatoid arthritis or prostate cancer.

Another aspect of the invention is a pharmaceutical composition comprising a therapeutically-effective amount of a compound according to any any of the above embodiments; and a pharmaceutically-acceptable diluent or carrier.

C_{Y-Z}alkyl, unless otherwise specified, means an alkyl chain containing a minimum Y total carbon atoms and a maximum Z total carbon atoms. These alkyl chains may be branched or unbranched, cyclic, acyclic or a combination of cyclic and acyclic. For example, the following substituents would be included in the general description “C₄₋₇alkyl”:

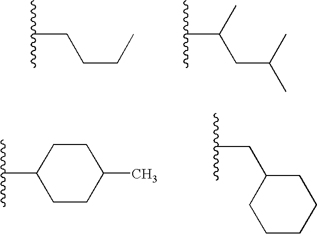

The term “oxo” means a double bonded oxygen (═O).

The compounds of the invention may contain heterocyclic substituents that are 5- or 6-membered ring heterocycles containing 1, 2 or 3 heteroatoms each independently selected from O, N and S and additionally having 0 or 1 oxo groups and 0 or 1 fused benzo rings. A nonexclusive list containing specific examples of such heterocycles are as follows: 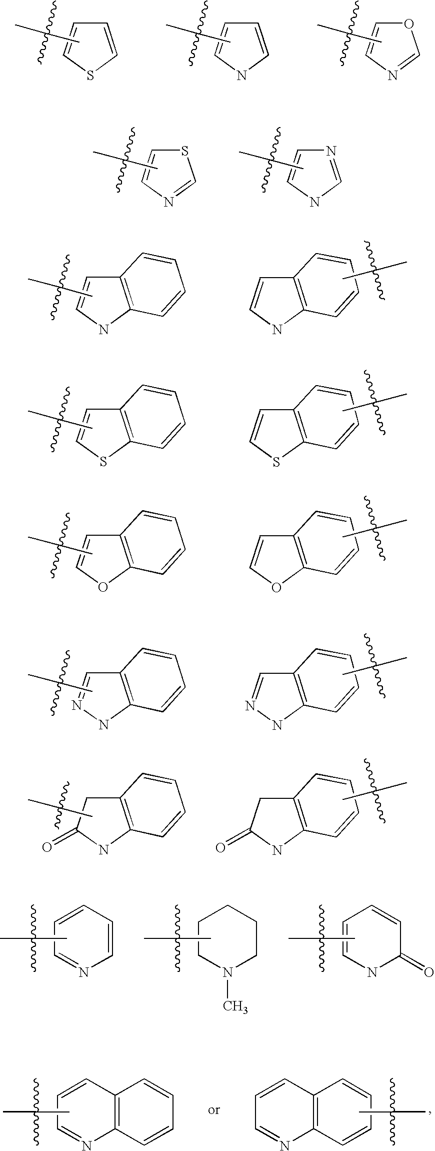
wherein the crossed bond represents that the heterocycle may be attached at any available position on either the heterocycle or the benzo ring.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bisulfate, butyrate, camphorate, camphorsulfonate, citrate, cyclohexyl sulfamate, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, 2-naphthalenesulfonate, nitrate, oxalate, pamoate, persulfate, phenylacetate, phosphate, picrate, pivalate, propionate, quinate, salicylate, stearate, succinate, sulfamate, sulfanilate, sulfate, tartrate, tosylate (p-toluenesulfonate), and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

Estrogen Receptor Binding Measurements

Abbreviated Procedure for Fluorescence Polarization Estrogen Receptor (ERFP) Binding Assay

A homogeneous mix-and-measure estrogen receptor (ER) binding assay which utilizes fluorescence polarization (FP) technology is used to identify compounds with affinity for the estrogen receptor. Purchased from PanVera (Madison, Wis.), assay reagents include purified human recombinant ERα, human recombinant ERβ, ES2 screening buffer (100 mM potassium phosphate, pH 7.4, 100 μg/mL bovine gamma globulin), and Fluormone™ ES2. Fluormone™ ES2, whose formulation is proprietary to PanVera, is a fluorescein-tagged, estrogen-like molecule which exhibits approximately equal affinity for ERα and ERβ.

For competition binding experiments, dilutions of test compounds are prepared at 2× the final assay concentration in 0.2% DMSO in ES2 Screening buffer on TECAN Genosys, and 25 μL compound/well is dispensed into black Costar ½ volume 96-well plates. Dependent upon a lot specific K_{d} determination, 10-40 nM ERα or 10-40 nM ERβ and 1 nM Fluormone ES2 are then added to these plates in a final assay volume of 50 μL/well. Plates are gently shaken for at least 5 minutes to mix and incubated for at least 1 hr 45 minutes to achieve equilibrium. (Reaction mixtures are stable for up to 5 hours). After centrifugation to remove air bubbles, plates are read on an LJL Analyst or Acquest equipped with Criterion software at the following settings: Fluorescence Polarization Mode; Static Polarizer on Excitation Side; Dynamic Polarizer on Emission Side; Excitation λ=485+/−10 nm; Emission λ=520+/−12:5 nm.

Polarized fluorescence intensity values are collected and subsequently converted electronically to millipolarization (mp) values. Following data reduction and normalization with Excel and/or Prism software, % Ctrl values at the various test concentrations are used to obtain IC₅₀ values via non-linear regression analysis of a four-parameter logistic equation.

Because ligand depletion is a consideration in this assay (˜40-60% input ES2 is bound in the assay), IC₅₀ values are converted to Kᵢ values through application of the Kenakin formula, as outlined in the reference below, rather than via the more routinely-used Cheng-Prusoff formula.
Reference: Bolger et al., Rapid Screening of Environmental Chemicals for Estrogen Receptor Binding Capacity, Environmental Health Pespectives:106 (1998), 1-7.
   Cell-Based Assay for ER Transcriptional Activity:

ERs are ligand-dependent transcription factors that bind the promoter regions of genes at a consensus DNA sequence called the estrogen responsive element (ERE). The ER agonist or antagonist activity of a drug was determined by measuring the amount of reporter enzyme activity expressed from a plasmid under the control of an estrogen-responsive element when cells transiently transfected with ER and the reporter plasmid were exposed to drug. These experiments were conducted according to the following methods.

Plasmids:

Estrogen Receptors alpha (αER, Gen Bank accession #M12674), and beta (βER, Gen Bank # X99101 were cloned into the expression vector pSG5 (Stratagene). A trimer of the vitellogenin-gene estrogen response element (vitERE) was synthesized as an oligonucleotide and attached to a beta-globin basal promoter in a construct named pERE3gal. This response element and promoter were removed from pERE3gal by digestion with the endonucleases SpeI (filled with Klenow fragment) and HindIII. This blunt/Hind III fragment was cloned into the β-galactosidase (β-gal) enhancer reporter plasmid (pBGALenh, Stratagene). αER and βER plasmids were purified using a the Endo Free Maxi Kit (Qiagen), and the DNA concentration and purity (A260/280 ratio) were determined spectrophotometrically (Pharmacia). Only DNA with A260/280 ratio of 1.8 and a concentration of >1 ug/uL was used for transfections.

Vitellogenin Response Element Sequence:

CTAGTCTCGAGAGGTCACTGTGACCT [symbol] AGGTCACTGTGACCTAGA
TCTAGGTCACTGTGACCTAC

= Spel overhang
= Xhol site
= AflII overhang
= ERE consensus
=

[symbol]

Cells:

All Transfections are performed in 293 cells (Human Embryonic Kidney cells ATCC # CRL-1573). Cells are grown in DMEM supplemented with 10% FBS, glutamine, sodium pyruvate and penicilin/streptomycin. Cells are grown to 70% confluency and split 1:4.

Transfection:

1. 293 cells are split the night before onto collagen I-coated 150 mm tissue-culture plates (Biocoat, Becton Dickinson #354551) at a density of 60-70% in DMEM (Mediatech 17-205-CV) 10% charcoal-stripped FBS (biocell #6201-31). Approximately 1×10⁷ cells/plate will yield 70% confluency.

2. The next morning, 1 hour prior to transfection, the media is changed to fresh DMEM 10% FBS stripped and supplements.

3. Transfections are performed using the Profection Kit (Promega #E1200). This kit is based on the calcium-phosphate-mediated transfection technique. Reagents are added in sterile polystyrene tubes in the following order:

Solution A
15 μg αER or βER
45 μg Reporter (pBGALenh or ERE3)
1.5 mL Sterile Water
186 μL CaCl₂
* Mix gently
Solution B
1.5 mL 2× Hank's Buffered Salt Solution
   4: Using a vortex set on low, add solution A to solution B dropwise. The resulting solution should become milky in color. It is important to achieve thorough mixing. The solution is allowed to settle for 30 minutes, then vortexed before adding the solution to cells.
   5. Add the mixture to 150 mm plates dropwise. Mix well by rocking plates back and forth and side to side gently. After an hour, a very fine precipitate should be seen floating on and above cells under 20× magnification. If this precipitate is not observed, the transfection will not be effective. Incubate the cells for 12 hours.
   Receptor Stimulation:
   1. The day after transfection, cells are washed 2× with calcium- and magnesium-free Mg free PBS containing 1 mM EGTA (pH 7.6). Cells are trypsinized for 2 min with 3 mL of typsin-EDTA. Trypsin is neutralized with DMEM 10% FCS. Cells are pelleted at 1000×g for 5 min. The cell pellet is then resuspended in 5 mL DMEM plus 2% phenol-red-free FCS supplemented with glutamine, pyruvate, and Penn/Strep.
   2. 50 μl of the resulting cell suspension is plated into each well of 96-well tissue culture dishes (Biocoat B&D #354407) using a multi-channel pipettor. The dishes have been previously loaded with 50 μL of DMSO-solubilized test compounds at twice the test concentration in DMEM. Data reported are either n=4 wells (single poke) and n=2 wells (9-point concentration-response curves).
   3. Cells are incubated overnight at 37° C. in the selected compounds.
   Reporter Assay:
   1. After 24 h, 100 μL of 7% CPRG (Roche 0884308) cocktail is added to each well in 1× Z-buffer, the plate is shaken gently at 37° C. for 3 h. CPRG turns bright red as it is cleaved by β-galactosidase.
   2. Absorbance measurments (570 nm) were obtained using a plate reader (Molecular Devices).

3. Data is compiled and analyzed using MS Excel.

10× Z Buffer

| | |
|---|---|
| Sodium Phosphate (dibasic) 1.7 g | 600 mM |
| Sodium Phosphate (monobasic) 0.96 g | 400 mM |
| Potassium Chloride 149 mg | 100 mM |
| Magnesium Sulfate 0.2 mL of 1 molar stock | 100 mM |
| BME 0.78 mL | 500 mM |
| Bring Final Volume to 20 mL with De-Ionized Water | |

### 7% CPRG Cocktail

For 50 mLs:

add 3.5 mL of 50 ml of CPRG

add 3.5 mL of 10× Z Buffer

add 1 mL of 10% SDS

bring to 50 mL with DI water

Typical Results:

Absorbance values illustrating typical concentration-response curves obtained for the ER agonist 17-β-estradiol (E) and the ER antagonist ICI182,780 (A) are plotted below for cells transfected with either αER or βER.

[image]

[image]

Administration and Use

Compounds of the present invention are shown to have high selectivity for ER-β over ER-α, and may possess agonist activity on ER-β without undesired uterine effects. Thus, these compounds, and compositions containing them, may be used as therapeutic agents in the treatment of various CNS diseases related to ER-β, such as, for example, Alzheimer's disease.

The present invention also provides compositions comprising an effective amount of compounds of the present invention, including the nontoxic addition salts, amides and esters thereof, which may, serve to provide the above-recited therapeutic benefits. Such compositions may also be provided together with physiologically-tolerable liquid, gel or solid diluents, adjuvants and excipients. The compounds of the present invention may also be combined with other compounds known to be used as therapeutic agents for the above or other indications.

These compounds and compositions may be administered by qualified health care professionals to humans in a manner similar to other therapeutic agents and, additionally, to other mammals for veterinary use, such as with domestic animals. Typically, such compositions are prepared as injectables, either as liquid solutions or suspensions; solid forms suitable for solution in, or suspension in, liquid prior to injection may also be prepared. The preparation may also be emulsified. The active ingredient is often mixed with diluents or excipients which are physiologically tolerable and compatible with the active ingredient. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

The compositions are conventionally administered parenterally, by injection, for example, either subcutaneously or intravenously. Additional formulations which are suitable for other modes of administration include suppositories, intranasal aerosols, and, in some cases, oral formulations. For suppositories, traditional binders and excipients may include, for example, polyalkylene glycols or triglycerides; such suppositories may be formed from mixtures containing the active ingredient. Oral formulations include such normally employed excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained-release formulations, or powders.

In addition to the compounds of the present invention that display ER-β activity, compounds of the present invention can also be employed as intermediates in the synthesis of such useful compounds.

Synthesis

Compounds within the scope of the present invention may be synthesized chemically by means well known in the art. The following Examples are meant to show general synthetic schemes, which may be used to produce many different variations by employing various commercially-available starting materials. These Examples are meant only as guides on how to make some compounds within the scope of the invention, and should not be interpreted as limiting the scope of the invention.

### EXAMPLES

The HPLC conditions used are the following unless stated otherwise: HPLC 4.6×50 mm C₁₈ 3 μm Alltech Rocket column; flow rate 2.0 mL/min, linear gradient from 10% B to 45% B over 2.0 min, 45% B to 70% B over 6 min; A=water, 0.05% TFA; B=acetonitrile, 0.05% TFA, UV detection at 254 nm.

DMF: N,N-dimethylformamide

THF: tetrahydrofuran

TFA: trifluoroacetic acid

DMSO: dimethylsulfoxide

### Example 1

### 6-Hydroxy-2-(4-hydroxyphenyl)-1-(2-phenethyl)-1H-benzimidazole

### 1) Synthesis of 2-fluoro-1-nitro-4-(2-trimethylsilylethoxymethoxy)benzene

To a solution of 3-fluoro-4-nitrophenol (3.2 g, 20 mmol) in dichloromethane (30 mL) was added a solution of diisopropylethylamine (3.7 g, 24 mmol) in dichloromethane (10 mL). To the resulting bright yellow solution was added 2-trimethylsilylethoxymethyl chloride (3.3 g, 20 mmol) dropwise and the mixture was stirred at room temperature for 72 h. The reaction was poured into dichloromethane and successively washed with saturated sodium bicarbonate and water. The organic phase was dried over MgSO₄, filtered and concentrated under vacuum to give a brown oil. This material was purified by bulb-to-bulb distillation (air bath temp 120° C., 0.1 mm Hg) to give the title compound (5.5 g, 96%) as a colorless oil. MS: 288 (MH⁺).

### 2) Synthetic method A: Synthesis of 2-nitro-N-(2-phenethyl)-5-(2-triethylsilylethoxymethoxy)aniline

To a solution of 2-fluoro-1-nitro-4-(2-trimethylsilylethoxymethoxy)benzene (1.4 g, 5 mmol) in THF (10 mL) was added phenethylamine (0.6 g, 5.0 mmol) and triethylamine (1.0 g, 10 mmol). The reaction was heated under reflux for 4 h then allowed to cool to room temperature. The reaction was diluted with dichloromethane and successively washed with saturated sodium bicarbonate and water. The organic phase was dried over MgSO₄, filtered and concentrated under vacuum to give the title compound (1.8 g) as a bright yellow oil which solidified on standing. MS: 389 (MH⁺).

### 3) Synthetic method B: Synthesis of N¹-(2-phenethyl)-5-(2-trimethylsilylethoxymethoxy)benzene-1,2-diamine

To a mixture of 2-nitro-N-(2-phenethyl)-5-(2-trimethylsilylethoxymethoxy)aniline (1.75 g, 4.5 mmol) and ammonium formate (1.42 g, 22.5 mmol) in absolute ethanol (50 mL) was added 10% palladium on carbon (0.24 g, 0.23 mmol). The mixture was heated under reflux for 3 h, then allowed to cool to room temperature and filtered through celite. The filter cake was washed with absolute ethanol and the combined filtrates concentrated under vacuum to give the title compound (1.3 g) as a dark colored viscous oil. NMR (DMSO-d₆): 7.39-7.21 (m, 5H), 6.48 (d, 1H, J=8.1 Hz), 6.23 (d, 1H, J=2.7 Hz), 6.13 (dd, 1H, J=8.1 Hz, J′=2.7 Hz), 5.06 (s, 2H), 3.70 (t, 2H, 3=8.1 Hz), 3.29-3.22 (m, 2H), 2.91 (t, 2H, J=8.1 Hz), 0.90 (t, 2H, 8.1 Hz), 0.01 (s, 9H); MS: 359 (MH⁺).

### 4) Synthetic method C: Synthesis of 2-(4-hydroxyphenyl)-1-(2-phenethyl)-6-(2-trimethylsilylethoxymethoxy)-1H-benzimidazole

To a solution of N¹-(2-phenethyl)-5-(2-trimethylsilylethoxymethoxy)benzene-1,2-diamine (0.25 g, 0.7 mmol) and ethyl 4-hydroxybenzimidate hydrochloride (0.12 g, 0.6 mmol) in absolute ethanol (20 mL) was added pyridine (0.22 g, 2.8 mmol). The mixture was heated under reflux for 2 h then allowed to cool to room temperature. The precipitated product was recovered as described in workup C1 below to give the title compound (110 mg). ¹H NMR (DMSO-d₆): 9.90 (s br, 1H), 7.54 (d, 1H, J=8.5 Hz), 7.41 (d, 2H, J=8.9 Hz), 7.32-7.17 (m, 4H), 7.05-6.99 (m, 2H), 6.95-6.86 (m, 3H), 5.31 (s, 2H), 4.42 (t, 2H, J=7.3 Hz), 3.79 (t, 2H, J=8.1 Hz), 2.99 (t, 2H, J=7.3 Hz), 0.97 (t, 2H, J=8.1 Hz), 0.02 (s, 9H); MS: 461 (MH⁺).

Workup C1: The precipitated product was collected by filtration, washed with hexane (five times) and dried under vacuum.

Workup C2: The reaction was diluted with ethyl acetate (30 mL) and successively washed with 0.2M hydrochloric acid (2×25 mL) and water. The organic phase was dried over sodium sulfate, filtered and concentrated under vacuum. The residue was purified by flash chromatography (eluant: 5% methanol in chloroform).

Workup C3: The reaction was diluted with ethyl acetate (30 mL) and successively washed with 0.2M hydrochloric acid (2×25 mL) and water. The solvent was removed under vacuum and the residue purified by HPLC (eluant: acetonitrile-water, gradient 25:75 to 90:10 over 40 minutes on a C18 column).

### 5) Synthetic method D: Synthesis of 6-hydroxy-2-(4-hydroxyphenyl)-1-(2-phenethyl)-1H-benzimidazole

A solution of 2-(4-hydroxyphenyl)-1-(2-phenethyl)-6-(2-trimethylsilylethoxymethoxy)-1H-benzimidazole (110 mg, 0.23 mmol) in methanol (5 mL) was treated with 1M hydrogen chloride in methanol. The resulting solution was stirred at room temperature for 30 min, then concentrated under vacuum. The residue was dried under vacuum to give the title compound (76 mg) as a purple solid. ¹H NMR (DMSO-d₆): 10.61 (s br, 1H), 10.15 (s br, 1H), 7.62 (d, 1H, J=8.9 Hz), 7.56-7.31 (m, 3H), 7.21-7.05 (m, 4H), 6.99 (d, 2H, J=8.9 Hz), 6.95-6.88 (m, 2H), 4.60 (t, 2H, J=6.9 Hz), 3.05 (t, 2H, J=6.9 Hz); MS: 331 (MH⁺); HPLC t_{R}: 2.32 min.

### Examples 2-27

Step 1: According to synthetic method A, from 2-fluoro-1-nitro-4-(2-trimethylsilylethoxymethoxy)benzene and the appropriate amines were obtained the following anilines:

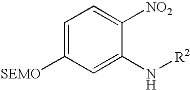

| R² | MS (MH⁺) |
|---|---|
| —Me | 297 (M − H)⁻ |
| —CH₂Ph | 373 (M − H)⁻ |
| —CH₂CH═CH₂ | |
| —CH₂CH₂CH₂CH₃ | 341 |
| —CH₂(2-thiophene) | 381 |
| —CH₂(4-Cl—Ph) | |
| —CH₂(4-F—Ph) | 393 |
| —CH₂CH₂(2-Cl—Ph) | 423 |
| —CH₂CH₂(2-thiophene) | 395 |
| —CH₂CH₂(3-Cl—Ph) | 387 (M − Cl)⁺ |
| —CH₂CH₂(3-MeO—Ph) | 419 |
| —CH₂CH₂(4-Cl—Ph) | 423 |
| —CH₂CH₂(4-Et—Ph) | 417 |
| —CH₂CH₂(4-F—Ph) | 407 |
| —CH₂CH₂(4-MeO—Ph) | 419 |
| —CH₂CH₂CH₂Ph | 403 |

Step 2: Synthetic method E: Synthesis of N¹-[2-(2-chlorophenyl)ethyl]-5-(2-trimethylsilylethoxymethoxy)benzene-1,2-diamine

In a 50 mL round bottom tube, equipped with a stir bar and pierceable cap with teflon lined silicon septum, sodium borohydride (0.23 g, 6.0 mmol) was added to a suspension of nickel(II) acetylacetonate (1.5 g, 6.0 mmol) in saturated ethanolic ammonia (10 mL). As the resulting mixture was stirred vigorously at room temperature for 10 min, the suspension slowly changed color from light green to gray-black accompanied with some gas evolution. A solution of N-[2-(2-chlorophenyl)ethyl]-2-nitro-5-(2-trimethylsilylethoxymethoxy)aniline (0.75 g, 1.8 mmol) in THF (3 mL) was added, accompanied by vigorous gas evolution. After the gas evolution ceased, the reaction vessel was capped and heated to 40° C. until the yellow color of the nitroaniline disappeared (from 5 to 45 minutes). The mixture was allowed to cool to room temperature and filtered through celite. The filter cake was washed with ethanol and the combined filtrates were concentrated under vacuum. The residue was purified by flash chromatography (eluant: hexane-ethyl acetate, gradient from 6:1 to 2:1) to give the desired product (370 mg) as a dark oil. MS: 393 (MH⁺).

The nitroanilines (from step 1) were reduced to the corresponding benzene-1,2-diamines according to synthetic methods B or E:

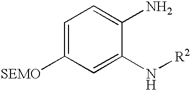

| R² | Synthetic Method | MS (MH⁺) |
|---|---|---|
| —Me | B | 269 |
| —CH₂Ph | B | 343 (M − H)⁻ |
| —CH₂CH═CH₂ and | E | 297* |
| —CH₂CH₂CH₃* | | |
| —CH₂CH₂CH₂CH₃ | B | 311 |
| —CH₂(2-thiophene) | E | 351 |
| —CH₂(4-Cl—Ph) | E | 379 |
| —CH₂(4-F—Ph) | B | 363 |
| —CH₂CH₂(2-thiophene) | E | 365 |
| —CH₂CH₂(3-Cl—Ph) | E | 393 |
| —CH₂CH₂(3-MeO—Ph) | B | 389 |
| —CH₂CH₂(4-Cl—Ph) | E | 393 |
| —CH₂CH₂(4-Et—Ph) | B | 387 |
| —CH₂CH₂(4-F—Ph) | B | 377 |
| —CH₂CH₂(4-MeO—Ph) | B | 389 |
| —CH₂CH₂CH₂Ph | B | 373 |

*reduction of N-allyl-2-nitro-5-(2-trimethylsilylethoxymethoxy)aniline gave a mixture of N-allyl and N-propylbenzene-1,2-diamine, due to partial reduction of the allyl group under these conditions; ion of m/z 297 assigned to the N-propyl compound.

Step 3: According to synthetic method C, the protected benzimidazoles were obtained after reaction between the corresponding benzene-1,2-diamine (from step 2) and the corresponding benzimidate.

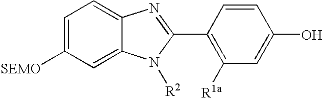

| R² | R^{1a} | Workup | MS (MH⁺) |
|---|---|---|---|
| —Me | H | C2 | 371 |
| —CH₂Ph | H | C1 | 447 |
| —CH₂CH₂CH₃ and | H | C1 | 399* |
| —CH₂CH═CH₂ | | | |
| —CH₂CH₂CH₂CH₃ | H | C2 | 413 |
| —CH₂(2-thiophene) | H | C1 | 453 |
| —CH₂(4-Cl—Ph) | H | C2 | 481 |
| —CH₂(4-F—Ph) | H | C1 | 465 |
| —CH₂CH₂(2-Cl—Ph) | H | C1 | 495 |
| —CH₂CH₂(2-thiophene) | H | C2 | 467 |
| —CH₂CH₂(3-Cl—Ph) | H | C1 | 495 |
| —CH₂CH₂(3-MeO—Ph) | H | C1 | 491 |
| —CH₂CH₂(4-Cl—Ph) | H | C1 | 495 |
| —CH₂CH₂(4-Et—Ph) | H | C2 | 489 |
| —CH₂CH₂(4-F—Ph) | H | C1 | 479 |
| —CH₂CH₂(4-MeO—Ph) | H | C1 | 491 |
| —CH₂CH₂CH₂Ph | H | C2 | 475 |
| —CH₂CH₂Ph | C1 | C1 | 495 |
| —CH₂CH₂CH₃ and | C1 | C3 | 433* |
| —CH₂CH═CH₂ | | | |
| —CH₂CH₂CH₂CH₃ | C1 | C2 | 447 |
| —CH₂CH₂(2-Cl—Ph) | C1 | C2 | 529 |
| —CH₂CH₂(2-thiophene) | C1 | C3 | 501 |
| —CH₂CH₂(3-MeO—Ph) | C1 | C2 | 525 |
| —CH₂CH₂(4-Et—Ph) | C1 | C2 | 523 |
| —CH₂CH₂(4-F—Ph) | C1 | C3 | 513 |
| —CH₂CH₂(4-MeO—Ph) | C1 | C3 | 525 |
| —CH₂CH₂CH₂Ph | C1 | C2 | 509 |

*ion assigned to the N-propyl compound.

Ethyl 2-chloro-4-hydroxybenzimidate hydrochloride was prepared as follows:

A mixture of 2-chloro-4-hydroxybenzaldehyde (1.0 g, 6.4 mmol) and hydroxylamine hydrochloride (0.8 g, 11.5 mmol) in dry N-methylpyrrolidinone (10 mL) was heated to 115° C. for 20 h. The reaction was cooled to room temperature and diluted with ethyl acetate and water. The organic phase was washed with water (five times), dried over MgSO₄ and filtered. The solvents were removed under vacuum and the residual solid purified by flash chromatography (eluant: hexane-ethyl acetate 4:1) to give 2-chloro-4-hydroxybenzonitrile (0.64 g) as a white solid, contaminated with 20% of 2-chloro-4-hydrobenzaldehyde oxime. ¹H NMR (CDCl₃): 7.53 (d, 1H, J=8.5 Hz), 6.93 (d, 1H, J=2.4 Hz), 6.81 (dd, 1H, J=8.5 Hz, J′=2.4 Hz).

A solution of 2-chloro-4-hydroxybenzonitrile obtained above (2.2 g, 14.4 mmol) in absolute ethanol (35 mL) was cooled to 0° C. in an ice/water bath. Anhydrous hydrogen chloride was passed through the solution until saturated. The resulting pink solution was stirred at room temperature for 66 h then the volatiles were removed under vacuum. The residual solid was triturated with ether (50 mL) and filtered. The filter cake was washed with either (10 mL) and dried under vacuum to give ethyl 2-chloro-4-hydroxybenzimidate hydrochloride (0.8 g, 23%) as a salmon colored solid. Concentration of the filtrate yielded 1.7 g of recovered starting material. ¹H NMR (DMSO-d₆): 11.56 (s br, 1H), 11.16 (s br, 1H), 7.64 (d, 1H, J=8.6 Hz), 7.05 (s, 1H), 6.94 (d, 1H, J=8.6 Hz), 4.56 (q, 2H, J=7.3 Hz), 1.43 (t, 3H, J=7.3 Hz).

Step 4: According to synthetic method D, the protected benzimidazoles (from step 3) were deprotected to give the corresponding benzimidazoles.

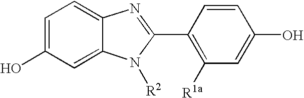

| Example | R² | R^{1a} | MS (MH⁺) | HPLC t_{R} (min) |
|---|---|---|---|---|
| 2 | —CH₂Ph | H | 317 | 2.24 |
| 3 | —Me | H | 241 | 1.51 |
| 4 | —CH₂CH₂CH₃ and | H | See Note (a) | |
| | —CH₂CH═CH₂ | | | |
| 5 | —CH₂CH₂CH₃ and | Cl | See Note (b) | |
| | —CH₂CH═CH₂ | | | |
| 6 | —CH₂CH₂CH₂CH₃ | H | 283 | 2.16 |
| 7 | —CH₂CH₂CH₂CH₃ | Cl | 317 | 2.35 |
| 8 | —CH₂(2-thiophene) | H | 323 | 2.15 |
| 9 | —CH₂(4-Cl—Ph) | H | 351 | 2.52 |
| 10 | —CH₂(4-F—Ph) | H | 335 | 2.32 |
| 11 | —CH₂CH₂(2-Cl—Ph) | H | 365 | 2.45 |
| 12 | —CH₂CH₂(2-Cl—Ph) | Cl | 399 | 2.63 |
| 13 | —CH₂CH₂(2-thiophene) | H | 337 | 2.22 |
| 14 | —CH₂CH₂(2-thiophene) | Cl | 371 | 2.40 |
| 15 | —CH₂CH₂(3-Cl—Ph) | H | 365 | 2.49 |
| 16 | —CH₂CH₂(3-MeO—Ph) | H | 361 | 2.09 |
| 17 | —CH₂CH₂(3-MeO—Ph) | Cl | 395 | 2.47 |
| 18 | —CH₂CH₂(4-Cl—Ph) | H | 365 | 2.53 |
| 19 | —CH₂CH₂(4-Et—Ph) | H | 359 | 2.70 |
| 20 | —CH₂CH₂(4-Et—Ph) | Cl | 393 | 2.85 |
| 21 | —CH₂CH₂(4-F—Ph) | H | 349 | 2.37 |
| 22 | —CH₂CH₂(4-F—Ph) | Cl | 383 | 2.53 |
| 23 | —CH₂CH₂(4-MeO—Ph) | H | 361 | 2.32 |
| 24 | —CH₂CH₂(4-MeO—Ph) | Cl | 395 | 2.49 |
| 25 | —CH₂CH₂CH₂Ph | H | 356 | 2.56 |
| 26 | —CH₂CH₂CH₂Ph | Cl | 379 | 2.70 |
| 27 | —CH₂CH₂Ph | Cl | 365 | 2.49 |
| (a) ratio: 4:1 N-propyl/N-allyl determined by HPLC/MS and NMR. | | | | |
| ¹H NMR (DMSO-d₆): non specific protons: 10.64(s br, 1H), 10.21(s br, 1H), 7.74-7.60(m, 3H), 7.29(d, 1H, J=2.1Hz), 7.14-7.05(m, 3H); allyl specific: 6.18-6.05(m, 1H, NCH₂CH═CH₂), 5.34(d, 1H, J=10.1Hz, cis-NCH₂CH═CH₂), 5.14(d, 1H, J=16.6Hz, trans-NCH₂CH═CH₂), 5.01-4.94(m, 2H, CH₂CH═CH₂); propyl specific: | | | | |
| #4.31(t, 2H, J=7.3Hz, NCH₂CH₂CH₃), 1.84-1.68(m, 2H, NCH₂CH₂CH₃), 0.80(t, 3H, J=7.3Hz, NCH₂CH₂CH₃). | | | | |
| N-propyl: HPLC t_{R}: 3.73 min; MS: 269 (MH⁺) and N-allyl: HPLC t_{R}: 3.54 min; MS: 267 (MH⁺); [in both cases, HPLC conditions are as follows: HPLC 2.1 × 50 mm C₈ 5 μm Zorbax Stablebond column; flow rate 0.7 mL/min; 5% B for 0.5 min, linear gradient from 5% B to 90% B over 9.5 min; A = water, 0.05% TFA; B = 90% acetonitrile, 10% water, 0.05% TFA, | | | | |
| #UV detection at 254 nm and positive ionization mass spectrometry detection] | | | | |
| (b) ratio: 3:1 N-propyl/N-allyl determined by HPLC/MS and NMR. | | | | |
| ¹H NMR (DMSO-d₆): non specific protons: 10.9(s br, 1H), 10.1(s br), 7.70-7.55(m, 2H), 7.25-7.21(m, 1H), 7.14-6.95(m, 3H); allyl specific: 5.93-5.79(m, 1H, NCH₂CH═CH₂), 5.19(d, 1H, J=10.9Hz, cis-NCH₂CH═CH₂), 5.08(d, 1H, J=17.7Hz, trans-NCH₂CH═CH₂), 4.76-4.70(m, 2H, CH₂CH═CH₂); propyl specific: | | | | |
| #4.10(t, 2H, J=6.9Hz, NCH₂CH₂CH₃), 1.70-1.58(m, 2H, NCH₂CH₂CH₃), 0.71(t, 3H, J=7.3Hz, NCH₂CH₂CH₃). | | | | |

N-propyl: HPLC t_{R}: 4.07 min; MS: 303 (MH⁺) and N-allyl: HPLC t_{R}: 3.93 min; MS: 3.01 (MH⁺); [HPLC conditions identical to those in note (a)]

### Example 28

### 6-Hydroxy-2-(4-hydroxyphenyl)-1-phenyl-1H-benzimidazole

### 1) Synthesis of 4-benzyloxy-2-fluoro-1-nitrobenzene

A mixture of 3-fluoro-4-nitrophenol (6.3 g, 40 mmol), benzyl bromide (8.2 g, 48 mmol) and potassium carbonate (8.4 g, 60 mmol) in DMF (100 mL) was stirred at room temperature for 48 h. The reaction was diluted with ether and washed with water. The organic layer was dried over MgSO₄, filtered and concentrated under vacuum. The residual solid was heated at 90° C. under vacuum (1 mm Hg) whereupon it melted and residual DMF and benzyl bromide distilled off. The residue was then purified by bulb-to-bulb distillation (air bath temp: ˜140° C./0.5 mm Hg) to give the title compound (8.9 g) as a yellow solid. MS: 248 (MH⁺)

### 2) Synthesis of 5-benzyloxy-2-nitro-N-phenylaniline

A solution of aniline (0.9 g, 10 mmol) in N-methylpyrrolidinone (5 mL) was added to sodium hydride (60% mineral oil suspension, 0.5 g, 12.5 mmol). The resulting mixture was stirred at room temperature for 45 min until all gas evolution had ceased; then a solution of 4-benzyloxy-2-fluoro-1-nitrobenzene (2.7 g, 11 mmol) in anhydrous N-methylpyrrolidinone (5 mL) was added. The resulting mixture was heated to 100° C. for 14 h then cooled to room temperature. The reaction was diluted with ethyl acetate then washed with water (five times). The combined aqueous washings were extracted twice with dichloromethane; the combined organic layers were dried over MgSO₄, filtered and concentrated under vacuum to a viscous oil. Purification by flash chromatography (eluant: hexane to 5% ethyl acetate in hexane) yielded the title compound (1.4 g) as a bright orange solid. MS: 321 (MH⁺)

### 3) Synthesis of 6-hydroxy-2-(4-hydroxyphenyl)-1-phenyl-1H-benzimidazole

To a solution of the above compound (0.8 g, 2.5 mmol) and ammonium formate (0.7 g, 10.5 mmol) in absolute ethanol (15 mL) was added 5% palladium on carbon (0.3 g, 0.13 mmol). The resulting mixture was heated at reflux for 2 h, allowed to cool then filtered through a pad of celite. The filter cake was washed with absolute ethanol (2×10 mL)) and the filtrates were combined. Ethyl 4-hydroxybenzimidate hydrochloride (0.5 g, 2.5 mmol) and pyridine (0.4 g, 5.0 mmol) were added. The resulting solution was heated at reflux for 14 h then cooled to room temperature. The solvents were removed under vacuum and the residue purified by flash chromatography (eluant: chloroform). Further purification by HPLC on a C18 column (eluting with acetonitrile-water, gradient from 0:100 to 45:55) gave the title compound (300 mg). ¹H NMR (DMSO-d₆): ¹H NMR (DMSO-d₆): 10.34 (br s, 1H), 9.78 (br s, 1H), 7.68-7.61 (m, 4H), 7.58-7.50 (m, 2H), 7.37 (d, 2H, J=8.3 Hz), 7.0-6.85 (m, 1H), 6.77 (d, 2H, J=8.3 Hz), 6.55-6.52 (m, 1H); MS: 303 (MH⁺)

### Example 29

### 1-Benzyl-5-hydroxy-2-(4-hydroxyphenyl)-1H-benzimidazole

### 1) Synthesis of N-(4-hydroxy-2-nitrophenyl)phthalimide

A suspension of 4-amino-3-nitrophenol (11.4 g) and phthalic acid (12.3 g) in acetic acid (120 mL) was heated at 100° C. for 18 h. The mixture was cooled. The solids were filtered, washed with water (three times) and methanol, and dried under high vacuum to give the title compound (13.1 g) as a pale yellow powder. ¹H NMR (DMSO-d₆): 10.90 (s, 1H), 8.0-7.9 (m, 4H), 7.56 (m, 2H), 7.31 (dd, 1H, J=8.7 Hz, J′=2.7 Hz).

### 2) Synthesis of N-(4-benzyloxy-2-nitrophenyl)phthalimide

A mixture of the above compound (10 g), benzyl bromide (8.4 mL), potassium carbonate (9.72 g) and potassium iodide (1 g) in DMF (100 mL) was stirred at room temperature for 6 h. The mixture was diluted with ethyl acetate, cooled at 0° C. and 5% hydrochloric acid was added slowly, until pH 6. The mixture was washed with water (three times). Evaporation of the solvents and trituration of the residue with ether-hexane gave the title compound (12.5 g). ¹H NMR (DMSO-d₆): 8.05-7.95 (m, 4H), 7.85 (d, 1H, J′=2.7 Hz), 7.70 (d, 1H, J=8.7 Hz), 7.60 (dd, 1H, J=8.7 Hz, J′=2.7 Hz), 7.60-7.35 (m, 5H), 5.31 (s, 2H).

### 3) Synthesis of 4-(benzyloxy)-2-nitroaniline

To a solution of the above compound (6.6 g) in THF (90 mL)-methanol (30 mL) was added hydrazine hydrate (2.56 g). The mixture was stirred at room temperature for 18 h and diluted with dichloromethane. The solids were filtered off and washed with dichloromethane. The filtrates were concentrated in vacuum and the residue was triturated with methanol. Filtration of the resulting solid afforded the title compound (3.83 g) as bright red crystals. ¹H NMR (CDCl₃): 7.66 (d, 1H, J=3 Hz), 7.35 (m, 5H), 7.14 (dd, 1H, J=9 Hz, J′=3 Hz), 6.76 (d, 1H, J=9 Hz), 5.89 (s br, 2H), 5.03 (s, 2H).

### 4) Synthesis of 4-benzyloxy-2 nitrotrifluoroacetanilide

To a solution of the above compound (2 g) and pyridine (2 mL) in dichloromethane (50 mL) cooled at 0° C. was added trifluoroacetic anhydride (1.5 mL) dropwise. The mixture was stirred at 0° C. for 1 h. 5% Hydrochloric acid was added and the mixture was extracted with dichloromethane. The organic layer was dried over MgSO₄ to give the title compound as a yellow powder (2.6 g). ¹H NMR (CDCl₃): 11.12 (s br, 1H), 8.63 (d, 1H, J=9 Hz), 7.86 (d, 1H, J=3 Hz), 7.40 (m, 6H), 5.15 (s, 2H).

### 5) Synthetic method F: Synthesis of N-benzyl-4-benzyloxy-2-nitroaniline

To a solution of 4-benzyloxy-2-nitrotrifluoroacetanilide (500 mg) in DMF (5 mL) was added benzyl bromide (524 μL, 3 eq.), potassium carbonate (1 g) and sodium iodide (100 mg). The mixture was stirred at room temperature for 6 h, poured into 5% hydrochloric acid and extracted with ethyl acetate. The organic layer was washed with water and brine, and dried over MgSO₄. The residue was purified by chromatography on a 10 g Bond Elute silica column (eluant: ethyl acetate-hexane, gradient from 0:100 to 20:80) to give N-benzyl-4-benzyloxy-2-nitrotrifluoroacetanilide (695 mg) as an oil.

To this compound (695 mg) in THF (10 mL) was added 1N sodium hydroxide (10 mL). The mixture was stirred at room temperature for 18 h, poured into ethyl acetate and water. The organic layer was washed with brine and dried over MgSO₄ to give the title compound (360 mg) as a red solid. ¹H NMR (CDCl₃): 8.36 (m, 1H), 7.75 (d, 1H, J=3 Hz), 7.35 (m, 10H), 7.15 (dd, 1H, J=9.3 Hz, J′=3 Hz), 6.78 (d, 1H, J=9.3 Hz), 5.02 (s, 2H), 4.55 (d, 2H, J=5.7 Hz).

### 6) Synthetic method G: Synthesis of N¹-benzyl-4-benzyloxybenzene-1,2-diamine

A solution of the above compound (360 mg) and tin(II) chloride dihydrate (1.2 g, 5 eq.) in ethyl acetate (10 mL) was refluxed for 1 h. The mixture was cooled, diluted with ethyl acetate and washed with 0.5N sodium hydroxide. The organic layer was washed with water and brine, and dried over MgSO₄ to give the title compound as an off-white solid (350 mg). ¹H NMR (DMSO-d₆): 7.30 (m, 10H), 6.29 (d, 1H, J=2.7 Hz), 6.24 (d, 1H, J=8.7 Hz), 6.06 (dd, 1H, J=8.7 Hz, J′=2.7 Hz), 4.88 (s, 2H), 4.68 (m, 3H), 4.21 (d, 2H, J=6 Hz).

### 7) Synthetic method H: Synthesis of 1-benzyl-5-benzyloxy-2-(4-hydroxyphenyl)-1H-benzimidazole

A solution of the above compound (150 mg) and ethyl 4-hydroxybenzimidate hydrochloride (100 mg, 1 eq) in ethanol (4 mL) was refluxed for 2 h. The mixture was cooled and the precipitate was filtered, washed with water and ether, and dried to give the title compound (115 mg). ¹H NMR (DMSO-d₆): 9.94 (s br, 1H), 7.6-7.2 (m, 12H), 7.0-6.8 (m, 5H), 5.51 (s, 2H), 5.14 (s, 2H); MS: 407 (MH⁺).

### 8) Synthesis of 1-benzyl-5-hydroxy-2-(4-hydroxyphenyl)-1H-benzimidazole

A mixture of the above compound (115 mg) and triethylsilane (400 μL) in trifluoroacetic acid (3 mL) was stirred at room temperature for 3 days, then heated at 55° C. for 30 min and at 70° C. for 30 min. The mixture was cooled and the solvents were evaporated in vacuo. Toluene (5 mL) was added and evaporated in vacuo. The residue was triturated with dichloromethane-ether to give the title compound as the trifluoroacetate salt (60 mg, pink solid). ¹H NMR (DMSO-d₆): 10.49 (s br, 1H), 9.97 (s br, 1H), 7.65 (d, 2H, J=8.7 Hz), 7.51 (d, 1H, J=9 Hz), 7.30 (m, 3H), 7.15-6.90 (m, 6H), 5.65 (s, 2H); MS: 317 (MH⁺); HPLC t_{R}: 2.34 min.

### Example 30

### 5-Hydroxy-2-(4-hydroxyphenyl)-1-methyl-1H-benzimidazole

1) From 4-benzyloxy-2-nitrotrifluoroacetanilide (500 mg) and methyl iodide, using synthetic methods F without sodium iodide, G and H, was obtained 5-benzyloxy-2-(4-hydroxyphenyl)-1-methyl-1H-benzimidazole (360 mg). MS: 331 (MH⁺).

2) Synthetic method I: Synthesis of 5-hydroxy-2-(4-hydroxyphenyl)-1-methyl-1H-benzimidazole

A mixture of 5-benzyloxy-2-(4-hydroxyphenyl)-1-methyl-1H-benzimidazole (150 mg) and triethylsilane (360 μL, 5 eq.) in trifluoroacetic acid (3 mL) was heated under reflux for 1 h. The solvents were evaporated in vacuo. Toluene (5 mL) was added, evaporated in vacuo and the residue was triturated with dichloromethane/ether to give the title compound as a pinkish solid (trifluoracetate salt, 114 mg). ¹H NMR (DMSO-d₆): 10.62 (s br, 1H), 10.11 (s br, 1H), 7.77 (m, 3H), 7.07 (m, 4H), 3.95 (s, 3H); MS: 241 (MH⁺); HPLC t_{R}: 1.56 min

### Example 31

### 5-Hydroxy-2-(4-hydroxyphenyl)-1-propyl-1H-benzimidazole

From 4-benzyloxy-2 nitrotrifluoroacetanilide and propyl iodide, using methods F (except that sodium iodide was not used and the mixture was stirred 18 h at room temperature and 30 min at 70° C. during the alkylation step), G, H and I, was obtained the title compound. MS: 269 (MH⁺); HPLC t_{R}: 2.00 min

### Example 32

### 5-Hydroxy-2-(4-hydroxyphenyl)-1H-benzimidazole

1) From 4-benzyloxy-2-nitroaniline was obtained 5-benzyloxy-2-(4-hydroxyphenyl)-1H-benzimidazole using methods G and H (except that in method H an aqueous work-up was used followed by an extraction with ethyl acetate). MS: 317 (MH⁺).

2) Synthesis of 5-hydroxy-2-(4-hydroxyphenyl)-1H-benzimidazole.

A mixture of the above compound (150 mg), 10% palladium on charcoal (100 mg) in ethanol (20 mL) was stirred under a 3 bar atmosphere of hydrogene for 3 h at room temperature. After filtration of the catalyst and evaporation of the solvents, the residue was dissolved in methanol and 4 drops of concentrated hydrochloric acid were added. The solvents were evaporated in vacuo and the residue triturated with ether to give the title compound (82 mg) as a solid (hydrochloride salt). ¹H NMR (DMSO-d₆): 10.76 (m, 1H), 10.07 (m, 1H), 8.10 (d, 2H, J=8.7 Hz), 7.58 (d, 1H, J=8.7 Hz), 7.05 (m, 4H); MS: 227 (MH⁺); HPLC t_{R}: 1.53 min

### Example 33

### 5-Hydroxy-2-(4-hydroxyphenyl)-1-phenyl-1H-benzimidazole

### 1) Synthesis of 4-benzyloxy-2-nitro-N-phenylaniline

A mixture of 4-benzyloxy-2-nitroaniline (1.4 g), potassium carbonate (1.2 g), copper powder (20 mg) in bromobenzene (5 mL) was heated at 165° C. for 18 h. The mixture was purified on a silica gel column (eluant: hexane, then dichloromethane-hexane (1:1)) to give the title compound (890 mg). ¹H NMR (CDCl₃): 9.35 (s br, 1H), 7.74 (d, 1H, J=3 Hz), 7.50-7.30 (m, 7H), 7.25-7.10 (m, 5H), 5.06 (s, 2H); MS: 321 (MH⁺).

2) From 4-benzyloxy-2-nitro-N-phenylaniline, according to methods G, H and I was obtained 5-hydroxy-2-(4-hydroxyphenyl)-1-phenyl-1H-benzimidazole. MS: 303 (MH⁺); HPLC t_{R}: 2.27 min.

Synthetic Method A1:

### N²-(2-thien-2-ylethyl)-4-(2-trimethylsilylethoxymethoxy)benzene-1,2-diamine

To a solution of 2-nitro-N-(2-thien-2-ylethyl)-5-(2-trimethylsilylethoxymethoxy)aniline (prepared by synthetic method A) (12.2 g, 3.0 mmol) and hydrazine monohydrate (12 mL, 248 mmol) in 95:5 ethanol:water (440 mL) was added 5% Ru/C (1.36 g, 0.67 mmol). The suspension was heated to 85 C for 1.5 h, then cooled to room temperature and filtered through celite. The filtrate was concentrated in vacuo, diluted with ethyl acetate and washed with water (8×25 mL). The organic phase was dried over Na₂SO₄, filtered and concentrated in vacuo to afford the title compound as a dark oil (11.1 g, 98%). ¹H NMR (DMSO-d₆): 7.36 (d, 1H, J=4.83 Hz), 6.99 (m, 2H), 6.48 (d, 1H, J=8.33 Hz), 6.21 (d, 1H, J=2.19 Hz), 6.14 (dd, 1H, J=7.89 Hz, 2.63 Hz), 5.05 (s, 2H), 4.68 (bt, 1H), 3.69 (t,2H, J=8.33 Hz), 3.28 (t, 2H, J=6.14), 3.11 (t, 2H, J=7.45), 0.9 (t, 2H, J=7.90 Hz).

### Synthetic Method A2: 6-Hydroxy-2-(4-methoxyphenyl)-1-(2-thien-2-ylethyl)-1-H-benzimidazole

To a solution of N²-(2-thien-2-ylethyl)-4-(2-trimethylsilylethoxymethoxy)benzene-1,2-diamine (0.69 g, 1.9 mmol) in absolute ethanol (13.8 mL) was added p-anisaldehyde (0.23 mL, 1.9 mmol). The solution was heated to 90 C for 20 h, then concentrated under vacuum. The residue was purified by reverse phase preperative HPLC (Method A3). Appropriate fractions were combined and concentrated in vacuo. The resulting material was diluted with methanol (8 mL) and trifluoroacetic acid (1.2 mL) was added. After heating at 50 C for 36 h, the dark solution was concentrated in vacuo. Purification of the deprotected product was accomplished by filtration through basic alumina, using ethyl acetate as eluent. The filtrate was concentrated in vacuo to afford the title compound as a light brown solid (0.17 g, 26%). ¹H NMR (DMSO-d₆): 9.70 (s br, 1H), 7.51 (dd, 3H, J=8.77 Hz, 2.63 Hz), 7.30 (d, 1H, J=5.26 Hz), 7.08 (d, 1H, J=8.77 Hz), 7.07 (s, 1H), 6.87 (m, 2H), 6.66 (d, 1H, J=3.06 Hz), 4.46 (t, 2H, J=6.58 Hz), 3.25 (t, 2H, J=6.58 Hz); MS: 351 (MH⁺).

Preparative HPLC Method A3: 20-95% (0.1% TFA-CH₃CN/0.1% TFA H₂O) over 30 min, Dynamax C18, 21.4 mm×250. Flow 15.0 mL/min, wavelength monitored: 220 nm.

Analytical HPLC Method A4: 1-99% 0.1% TFA-CH₃CN/0.1% TFA H₂O over 7.5 m, Zorbax C8, 3.5 um, 3.0 mm×150 mm. Flow 0.8 mL/m, wavelengths monitored: 220, 254, 280 nm.

| Example | Structure | Synthetic Methods | MS (MH+) | HPLC (Method A4) |
|---|---|---|---|---|
| 34 | 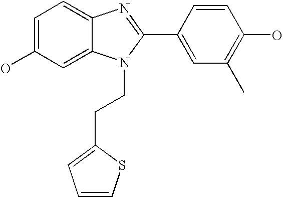 | A1, A2 | 351 | 5.88 |
| 35 | 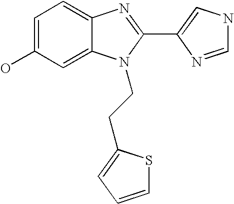 | A1, A2 | 311 | 5.09 |
| 36 | 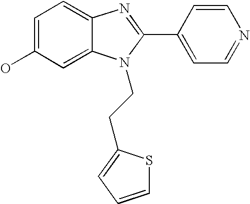 | A1, A2 | 322 | 4.85 |
| 37 | 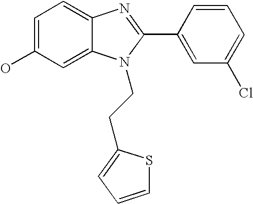 | A1, A2 | 355 | 6.26 |
| 38 | 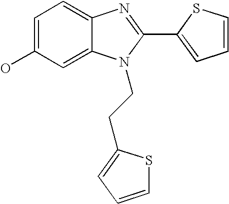 | A1, A2 | 327 | 5.81 |
| 39 | 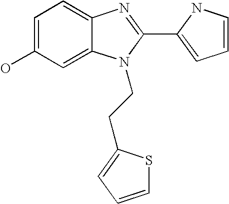 | A1, A2 | 310 | 5.78 |
| 40 | 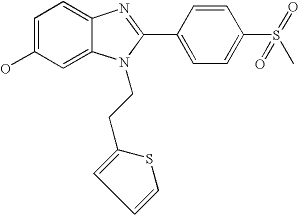 | A1, A2 | 399 | 5.52 |
| 41 | 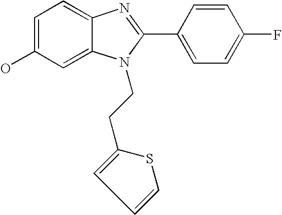 | A1, A2 | 339 | 6.07 |
| 42 | 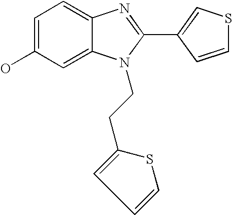 | A1, A2 | 327 | 5.84 |
| 43 | 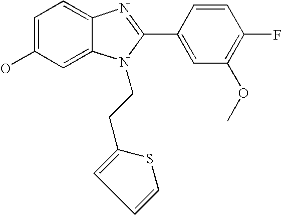 | A1, A2 | 369 | 6.19 |
| 44 | 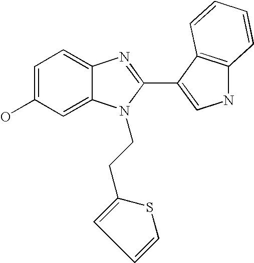 | A1, A2 | 360 | 6.05 |
| 45 | 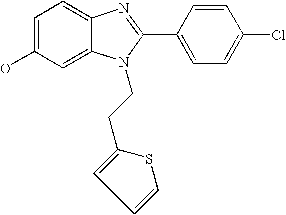 | A1, A2 | 355 | 6.27 |
| 46 | 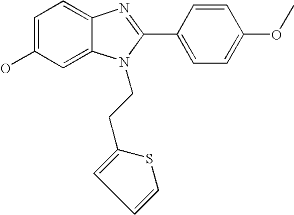 | A1, A2 | 351 | 6.24 |

The approximate activity and selectivity ranges for the benzimidazoles exemplified in this specification are as follows:

| | Activity (nM) | Selectivity (ERβ/ERα) |
|---|---|---|
| FP (binding assay) | 15 → 2000 | 30 → 0.5 |
| ERE (functional assay) | 1 → 1200 | 250 → 0.005 |

1. A compound of the formula: 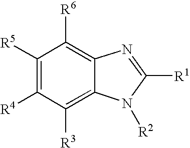
or a pharmaceutically acceptable salt or hydrolyzable ester thereof,
wherein:
R¹ is C₁₋₈alkyl, phenyl, benzyl or a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈alkyl, phenyl, benzyl or heterocycle is substituted by 1, 2 or 3 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; and wherein the phenyl, benzyl or heterocycle is additionally substituted by 0, 1 or 2 substituents selected from C₁₋₆alkyl, phenyl and benzyl;
R² is H, C₁₋₆alkyl, —(CH₂)ₘphenyl, —(CH₂)ₘnaphthyl or —(CH₂)ₘheterocycle, wherein the heterocycle is a 5- or 6-membered ring heterocycle containing 1, 2 or 3 heteroatoms each independently selected from O, N and S and additionally having 0 or 1 oxo groups and 0 or 1 fused benzo rings, wherein the C₁₋₆alkyl, —(CH₂)ₘphenyl, —(CH₂)ₘnaphthyl or —(CH₂)ₘheterocycle is substituted with 0, 1 or 2 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl;
R³ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl; or R³ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;
R⁴ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —R^{a}S(═O)₂R^{a}, —C(═O)R^{a}, —S(═O)R^{a}, —S(═O)₂R^{a}, halogen, cyano, nitro or C₁₋₃haloalkyl;
R⁵ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl;
R⁶ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl; or R⁶ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;
R^{a} is H, C₁₋₆alkyl, C₁₋₃haloalkyl, phenyl or benzyl; and
m is 0, 1, 2 or 3.

2. The compound according to claim 1, wherein R¹ is C₁₋₈alkyl or a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈alkyl or heterocycle is substituted by 0, 1, 2 or 3 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; and wherein the heterocycle is additionally substituted by 0, 1 or 2 substituents selected from C₁₋₆alkyl, phenyl and benzyl;

3. The compound according to claim 1, wherein R² is C₁₋₆alkyl, —(CH₂)ₘphenyl, —(CH₂)ₘnaphthyl or —(CH₂)ₘheterocycle, wherein the heterocycle is a 5- or 6-membered ring heterocycle containing 1, 2 or 3 heteroatoms each independently selected from O, N and S and additionally having 0 or 1 oxo groups and 0 or 1 fused benzo rings, wherein the —(CH₂)ₘphenyl, —(CH₂)ₘnaphthyl or —(CH₂)ₘheterocycle is substituted with 0, 1 or 2 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; and the C₁₋₆alkyl is substituted with 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

4. The compound according to claim 1, wherein R³ is C₁₋₆alkyl, —OR^{a}, —SR^{a}, —NR^{a}R^{a}, —CO₂R^{a}, —OC(═O)R^{a}, C(═O)NR^{a}R^{a}, —NR^{a}C(═O)R^{a}, —NR^{a}S(═O)R^{a}, —NR^{a}S(═O)₂R^{a}—C(═O)R^{a}, —S(═O)R^{a}, —S(═O)₂R^{a}, halogen, cyano, nitro end or C₁₋₃haloalkyl; or R³ is C₁₋₃alkyl containing 1 or 2-substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

5. The compound according to claim 1, wherein R⁴ is —R^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl.

6. The compound according to claim 1, wherein R⁵ is —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl.

7. The compound according to claim 1, wherein R⁶ is C₁₋₆alkyl, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}; —S(═O)R^{a}, —S(═O)₂R^{a}, halogen, cyano, nitro or C₁₋₃haloalkyl; or R⁶ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}—NR^{a}(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

8. The compound according to claim 1, wherein R¹ is phenyl or benzyl, wherein the phenyl or benzyl is substituted by 1, 2 or 3 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; and wherein the phenyl or benzyl is additionally substituted by 0, 1 or 2 substituents selected from C₁₋₆alkyl, phenyl and benzyl.

9. The compound according to claim 8, wherein R¹ is 4-hydroxyphonyl substituted by 0, 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)₂R^{a}, —NR^{a}S(═O)₂R², —C(═O)R^{a}, —S(═O)R^{a}, —S(═O)₂R^{a}, halogen, cyano, nitro and C₁₋₃haloalkyl.

10. The compound according to claim 1, wherein R⁴ is OH.

11. The compound according to claim 1, wherein R⁵ is OH.

12. The compound according to claim 9, wherein R⁴ is OH.

13. The compound according to claim 9, wherein R⁵ is OH.

14. The compound according to any one of claims 1-13, wherein the compound satisfies the equation:
(K_{iαA}/K_{iβA})/(K_{iαE}/K_{iβE})>30, wherein
K_{iαA} is the Kᵢ value for the agonist in ER-α;
K_{iβA} is the Kᵢ value for the agonist in ER-β;
K_{iαE} is the Kᵢ value for estrogen in ER-α; and
K_{iβE} is the Kᵢ value for estrogen in ER-β.

15. (canceled)

16. A method for the treatment or prophylaxis of Alzheimer's disease, anxiety disorders, depressive disorders, osteoporosis, cardiovascular disease, rheumatoid arthritis or prostate cancer, which comprises administering to a patient in need of such treatment or prophylaxis an effective amount of a compound according to any one of claims 1-13.

17. A pharmaceutical composition comprising:
a therapeutically-effective amount of a compound according to any one of claims 1-13; and
a pharmaceutically-acceptable diluent or carrier.

18. A method for the treatment or prophylaxis of Alzheimer's disease, anxiety disorders, depressive disorders, osteoporosis, cardiovascular disease, rheumatoid arthritis or prostate cancer, which comprises administering to a patient in need of such treatment or prophylaxis an effective amount of a compound according to claim 14.

19. A pharmaceutical composition comprising:
a therapeutically-effective amount of a compound according to claim 14; and
a pharmaceutically-acceptable diluent or carrier.
